(19) United States
(12) Patent Application Publication
(10) Pub. No.: US 2021/0236000 A1
(43) Pub. Date: Aug. 5, 2021

(54) METHOD, DEVICE AND SYSTEM FOR ACQUIRING BLOOD VESSEL EVALUATION PARAMETERS BASED ON ANGIOGRAPHIC IMAGE

(71) Applicant: SUZHOU RAINMED MEDICAL TECHNOLOGY CO., LTD., Suzhou (CN)

(72) Inventors: Yunfei HUO, Suzhou (CN); Guangzhi LIU, Suzhou (CN); Xingyun WU, Suzhou (CN)

(73) Assignee: SUZHOU RAINMED MEDICAL TECHNOLOGY CO., LTD., Suzhou (CN)

(21) Appl. No.: 17/237,662

(22) Filed: Apr. 22, 2021

Related U.S. Application Data
(63) Continuation of application No. PCT/CN2019/118053, filed on Nov. 13, 2019.

(30) Foreign Application Priority Data
Nov. 13, 2018 (CN) 201811344060.X
Nov. 13, 2018 (CN) 201811344074.1
Nov. 13, 2018 (CN) 201811344281.7

(51) Int. Cl. A61B 5/02; A61B 5/00; A61B 5/0215; A61B 5/0275; G06T 7/00; G06T 17/20; G16H 50/30; G16H 30/40; G16H 50/50

(52) U.S. Cl. CPC A61B 5/02007; A61B 5/7257; A61B 5/0215; A61B 5/0275; G06T 2207/30101; G06T 17/20; G16H 50/30; G16H 30/40; G16H 50/50; G06T 7/0012

(57) ABSTRACT

A method, device and system for acquiring blood vessel evaluation parameters based on an angiographic image are provided. The method includes: acquiring a real-time pressure Pₐ at a coronary artery inlet in an angiographic state to obtaining a Pₐ-t pressure waveform in time domain; subjecting a segment of blood vessel of interest in a two-dimensional angiographic image of the coronary artery in the angiographic state to three-dimensional modeling to obtain a three-dimensional grid model for the blood vessel; acquiring a real-time blood flow velocity v of the three-dimensional grid model for the blood vessel to obtain a v-t velocity waveform in the time domain; obtaining a ΔP-t pressure drop waveform in the time domain from the coronary artery inlet to a distal end of the coronary artery stenosis through Fourier transform and the inverse Fourier transform; acquiring the coronary artery blood vessel evaluation parameters in the angiographic state.

### CROSS-REFERENCE TO RELATED APPLICATION

This application is a continuation of International Application No. PCT/CN2019/118053, filed on Nov. 13, 2019 which is based upon and claims priority to Chinese Patents Applications 201811344060.X filed Nov. 13, 2018, 201811344074.1 filed Nov. 13, 2018, and 201811344281.7 filed Nov. 13, 2018, the entire contents of which are hereby incorporated by reference.

### TECHNICAL FIELD

The disclosure relates to the technical field of coronary artery, and in particular to, a method, a device, a coronary artery analysis system and a computer storage medium for acquiring coronary artery blood vessel evaluation parameters based on an angiographic image.

### BACKGROUND

According to statistics from the World Health Organization, cardioblood vessel diseases have become the “top killer” of human health. In recent years, analysis of the physiology and pathological behaviors of the cardioblood vessel diseases using hemodynamics has also become a very important method for the diagnosis of the cardioblood vessel diseases.

Fractional flow reserve (FFR) may indicate an influence of coronary artery stenosis lesion on distal blood flow and diagnosis of myocardial ischemia, and thus has become a recognized indicator of the functional evaluation of the coronary artery stenosis. FFR is defined as a ratio of the maximum blood flow to the myocardium in the innervated region provided by the coronary artery stenosis to the maximum blood flow to the myocardium provided by the same normal coronary artery. The FFR may be simplified as a ratio of a mean intracoronary pressure (P_{d}) at a distal end of the stenosis in a myocardium's maximum hyperemia state to a mean aortic pressure (Pₐ) at a coronary artery inlet, that is, FFR=Pd/Pa.

When determining FFR, it is necessary to calculate FFR based on a blood flow velocity and the mean aortic pressure (Pₐ) at the coronary artery inlet in the myocardium's maximum hyperemia state, and the mean intracoronary pressure at the distal end of the stenosis obtained by different technical means. However, intracoronary injection or intravenous injection of adenosine or ATP is required for maximum hyperemia of the myocardium, and the injection of adenosine or ATP will decrease the aortic pressure and will cause a certain side effects, such as atrioventricular block, sinus bradycardia, and sinus arrest, and contraindications include second degree atrioventricular block, third degree atrioventricular block, sinoatrial node diseases, tracheal or bronchial asthma, and adenosine allergy.

Instantaneous wave-free ratio (iFR) can provide a method for measuring intracoronary pressure similar to Fractional Flow Reserve (FFR). iFR does not require a vasodilator, is easy to operate, and will be more frequently used in coronary artery interventional therapy.

At present, the existing measurement methods for coronary artery blood vessel evaluation parameters are mainly: (1) measurement by pressure guide wire, which is expensive, difficult and risky; (2) measurement through two-dimensional coronary artery angiographic images by obtaining a real-time aortic pressure Pₐ and the measured full-cycle blood flow velocity v, resulting in obtaining only full-cycle coronary artery blood vessel evaluation parameters instead of blood vessel evaluation parameters in a diastolic phase in an angiographic state; and there is no screening of the aortic pressure Pₐ and the blood flow velocity v, resulting in inaccurate blood vessel evaluation parameters.

### SUMMARY

The disclosure provides a method, device and system for acquiring blood vessel evaluation parameters based on an angiographic image, which solve the problems of being unable to obtain blood vessel evaluation parameters during diastolic phase and inaccurate measured blood vessel evaluation parameters by obtaining Pₐ-t pressure wave and v-t velocity waveform in time domain.

In order to achieve the above objectives, in a first aspect, the disclosure provides a method for acquiring coronary artery blood vessel evaluation parameters based on an angiographic image, comprising:

acquiring a real-time pressure Pₐ at a coronary artery inlet in an angiographic state to obtain a Pₐ-t pressure waveform in time domain:

subjecting a segment of blood vessel of interest in a two-dimensional angiographic image of the coronary artery in an angiographic state to three-dimensional modeling to obtain a three-dimensional grid model for the blood vessel;

acquiring a real-time blood flow velocity v of the three-dimensional grid model for the blood vessel to obtain a v-t velocity waveform in the time domain;

subjecting Fourier transform to the blood flow velocity v and pressure Pₐ to obtain v′-t velocity waveform and Pₐ′-t pressure waveform in frequency domain;

acquiring a ΔP′-t pressure drop waveform in the frequency domain from the coronary artery inlet to a distal end of the coronary artery stenosis based on the v-t velocity waveform and Pₐ′-t pressure waveform in the frequency domain:

acquiring the ΔP-t pressure drop waveform from the coronary artery inlet to the distal end of the coronary artery stenosis in the time domain through the inverse Fourier transform;

acquiring the coronary artery blood vessel evaluation parameters in the angiographic state based on the Pₐ-t pressure waveform and the ΔP-t pressure drop waveform in the time domain.

Optionally, in the above method for acquiring the coronary artery blood vessel evaluation parameters based on the angiographic image, the blood vessel evaluation parameters comprise: instantaneous wave-free ratio caiFR in the angiographic state, a first diastolic phase pressure ratio in the angiographic state cadPR and a ratio of diastolic phase less than the average pressure in the angiographic state caDFR.

Wherein, the first diastolic phase pressure ratio cadPR is obtained through the first mean pressure of aorta P̅ₐ̅₁̅ in the full diastolic phase and the first mean pressure of a distal end of artery stenosis P̅_̅{̅d̅1̅}̅.

The ratio of diastolic phase less than the average pressure caDFR is obtained through the second mean pressure of aorta P̅ₐ̅₂̅ and the second mean pressure of a distal end of coronary artery stenosis P̅_̅{̅d̅2̅}̅ within an interval from Pₓ<P̅ₐ̅₁̅ to the aortic pressure Pₙ=Pₘᵢₙ, wherein Pₘ and Pₙ indicate Pₘᵢₙ and nth aortic pressures during the diastolic phase, respectively, and Pₘᵢₙ indicates the minimum aortic pressure.

The instantaneous wave-free ratio caiFR is obtained through the third mean pressure of aorta P̅ₐ̅₃̅ and the third mean pressure of a distal end of coronary artery stenosis P̅_̅{̅d̅3̅}̅ within the period of wave-free.

Optionally, in the above method for acquiring the coronary artery blood vessel evaluation parameters based on the angiographic images, calculation formulas of the instantaneous wave-free ratio caiFR, the first diastolic pressure ratio cadPR and the ratio of diastolic phase less than the average pressure caDFR each are:

cadPR=P̅_̅{̅d̅1̅}̅/P̅ₐ̅₁̅;caDFR=P̅_̅{̅d̅2̅}̅/P̅ₐ̅₂̅;caiFR=P̅_̅{̅d̅3̅}̅/P̅ₐ̅₃̅.

Optionally, in the above method for acquiring coronary artery blood vessel evaluation parameters based on the angiographic images, the blood vessel evaluation parameters further comprise: fractional flow reserve caFFR.

Wherein, fractional flow reserve caFFR is obtained through the fourth mean pressure of aorta P̅ₐ̅₄̅ and the fourth mean pressure of a distal end of coronary artery stenosis P̅_̅{̅d̅4̅}̅ in the whole cardiac cycle, namely:

caFFR=P̅_̅{̅d̅4̅}̅/P̅ₐ̅₄̅.

Optionally, in the above method for acquiring the coronary artery blood vessel evaluation parameters based on angiographic images, the method for acquiring the real-time pressure Pₐ at the coronary artery inlet in the angiographic state to obtain the Pₐ-t pressure waveform in the time domain comprises: real-time measuring the pressure Pₐ at the coronary artery inlet through a blood pressure acquisition device connected with a catheter for angiography, and drawing a curve with time to obtain the Pₐ-t pressure waveform in the time domain.

Optionally, in the above method for acquiring the coronary artery blood vessel evaluation parameters based on the angiographic images, the method for subjecting the segment of the blood vessel of interest in the two-dimensional angiographic image of the coronary artery in the angiographic state to three-dimensional modeling to obtain the three-dimensional grid model for the blood vessel comprises:

reading at least two coronary artery angiographic images with different angles;

subjecting a certain segmented blood vessel on the two coronary angiographic images with a mapping relationship to a 3D reconstruction through 2D structure data to obtain a 3D model and 3D data of the segmented blood vessel;

repeating the above steps until the three-dimensional reconstructions of all the segmented vessels are completed, and then merging all the reconstructed segmented vessels to obtain a complete three-dimensional grid model for the blood vessel.

Optionally, in the above method for acquiring the coronary artery blood vessel evaluation parameters based on the angiographic images, the method for acquiring the real-time blood flow velocity v of the three-dimensional grid model for the blood vessel to obtain a v-t velocity waveform in the time domain comprises: v-t velocity waveform in the time domain in the diastolic phase;

acquiring a start point of the diastolic phase from an image corresponding to a two-dimensional starting frame and an end point of the diastolic phase from an image corresponding to a two-dimensional end frame, respectively; and taking a length L of the blood vessel in the diastolic phase from three-dimensional grid model for the blood vessel by using the start point and the end point;

acquiring a length difference for a flow of a contrast agent in the blood vessel between two adjacent frames of images according to a patient's heart rate and the number of frames used for the coronary artery angiographic images in a certain diastolic phase, and acquiring the contrast agent speed of each frame of the coronary artery angiographic image, namely the real-time blood flow velocity v in the diastolic phase of the three-dimensional grid model for the blood vessel, the specific formula being as follows:

$$v = Δ L / [ \frac{K ( 60 / H )}{y} ] ;$$

$$K = t / T ,$$

wherein, ΔL represents the length difference for the flow of the contrast agent in the blood vessel between two adjacent frames of images, H represents the patient's heart rate with a unit of beats/min; y represents the number of frames of the coronary artery angiographic images in the diastolic phase, wherein t represents the time for the diastolic phase in the Pₐ-t pressure waveform, T represents the time for the whole cardiac cycle in the Pₐ-t pressure waveform:

plotting the blood flow velocity v against time to obtain the v-t velocity waveform in the time domain in the diastolic phase.

Optionally, the above method for acquiring the coronary artery blood vessel evaluation parameters based on the angiographic image, the method for acquiring the real-time blood flow velocity v of the three-dimensional grid model for blood vessel to obtain the v-t velocity waveform in the time domain comprises: v-t velocity waveform in the time domain in the whole cardiac cycle;

acquiring a start point of the cardiac cycle from an image corresponding to a two-dimensional starting frame and an end point of the cardiac cycle from an image corresponding to a two-dimensional end frame, respectively: and taking a length L of the blood vessel within one cardiac cycle from three-dimensional grid model for the blood vessel by using the start point and the end point:

acquiring a length difference for a flow of a contrast agent in the blood vessel between two adjacent frames of images according to a patient's heart rate and the number of frames used for the coronary artery angiographic images in a certain cardiac cycle, and acquiring the contrast agent speed of each frame of the coronary artery angiographic image, namely the real-time blood flow velocity v in the whole cardiac cycle of the three-dimensional grid model for the blood vessel, the specific formula being as follows:

$$v = Δ L / [ \frac{K ( 60 / H )}{y} ] ;$$

wherein, ΔL represents the length difference for the flow of the contrast agent in the blood vessel between two adjacent frames of images H represents the patient's heart rate with a unit of beats/min, and x represents the number of the frames of the coronary artery angiographic images during the cardiac cycle;

plotting the blood flow velocity v against time to obtain the v-t velocity waveform in the time domain in the cardiac cycle.

Optionally, in the above method for acquiring the coronary artery vessel evaluation parameters based on the angiographic image, the method for acquiring a real-time blood flow velocity v of the three-dimensional grid model for blood vessel comprises:

v=ΔL/fps;

wherein, ΔL represents the length difference for the flow of the contrast agent in the blood vessel between two adjacent frames of images and fps represents the number of frames transmitted per second.

Optionally, in the above method for acquiring the coronary artery blood vessel evaluation parameters based on the angiographic image, the method for acquiring the ΔP′-t pressure drop waveform in the frequency domain from the coronary artery inlet to the distal end of the coronary artery stenosis based on the v′-t velocity waveform and Pₐ′-t pressure waveform in the frequency domain comprises:

use numerical methods to solve the continuity and using Navier-Stokes equation to calculate the pressure drop ΔP′ from the inlet to various points in the downstream along the center line of blood vessels, an inlet boundary condition being the blood flow velocity v′ in the frequency domain, and an outlet boundary condition being the out-flow boundary condition;

wherein, the continuity and Navier-Stokes equation being:

$$▽ \cdot \vec{V} = 0 ; \\ ρ \frac{\partial \vec{V}}{\partial t} + ρ \vec{V} \cdot ▽ \vec{V} = - ▽ P + ▽ \cdot {μ ( ▽ \vec{V} + ▽ \vec{V} )}^{T} ) ;$$

\ddot{V}, P, ρ, μ represent a coronary artery blood flow velocity, a pressure, a blood flow density, and a blood flow viscosity, respectively;

plotting the pressure drop ΔP′ against time to obtain the ΔP′-t pressure drop waveform in the frequency domain.

In the second aspect, the disclosure provides a device for acquiring coronary artery blood vessel evaluation parameters based on an angiographic image and the device is used in the method for acquiring coronary artery blood vessel evaluation parameters based on angiographic images, the device comprises: a blood pressure acquisition device, and a three-dimensional modeling unit, a blood flow velocity unit, a pressure drop unit, and a coronary artery blood vessel evaluation parameter unit connected in sequence, and the blood pressure acquisition device being connected to the pressure drop unit.

The blood pressure acquisition device is connected with an external catheter for angiography and configured for acquiring a real-time pressure Pₐ at a coronary artery inlet in an angiographic state to obtain a Pₐ-t pressure waveform in time domain.

The three-dimensional modeling unit is configured for reading the coronary artery angiographic image in the angiographic state, and subjecting a segment of blood vessel of interest in the image to three-dimensional modeling to obtain a three-dimensional grid model for the blood vessel.

The blood flow velocity unit is configured for receiving the three-dimensional grid model for the blood vessel sent by the three-dimensional modeling unit and acquiring a real-time blood flow velocity v of the segment of blood vessel of interest to obtain v-t velocity waveform in the time domain.

The pressure drop unit is configured for receiving the v-t velocity waveform and the Pₐ′-t pressure waveform in the time domain sent by the blood flow velocity unit and the blood pressure acquisition device, respectively, and subjecting the blood flow velocity v and pressure Pₐ to Fourier transform to obtain v′-t velocity waveform and Pₐ′-t pressure waveform in frequency domain; then acquiring a ΔP′-t pressure drop waveform in the frequency domain from the coronary artery inlet to the distal end of the coronary artery stenosis according to the v′-t velocity waveform and Pa′-t pressure waveform in the frequency domain; acquiring the ΔP-t pressure drop waveform from the coronary artery inlet to the distal end of the coronary artery stenosis in the time domain through the inverse Fourier transform.

The coronary artery blood vessel evaluation parameter unit is configured for receiving the Pₐ-t pressure waveform and the ΔP-t pressure drop waveform in the time domain sent by the blood pressure acquisition device and the pressure drop unit, and acquiring the coronary artery blood vessel evaluation parameters.

Optionally, in the above device for obtaining the coronary artery blood vessel evaluation parameters based on the angiographic image, the coronary artery vessel evaluation parameter unit further comprises: a caiFR module, a cadPR module, a caDFR module, and a caFFR module.

The cadPR module is configured to obtain the first diastolic phase pressure ratio cadPR through the first mean pressure of aorta P̅ₐ̅₁̅ in the full diastolic phase and the first mean pressure of a distal end of artery stenosis P̅_̅{̅d̅1̅}̅.

The caDFR module is configured to obtain the ratio of a diastolic phase less than the average pressure caDFR through the second mean pressure of aorta P̅ₐ̅₂̅ and the second mean pressure of a distal end of coronary artery stenosis P̅_̅{̅d̅2̅}̅ within an interval from pₘ<P̅ₐ̅₁̅ to the aortic pressure Pₙ=Pₘᵢₙ wherein Pₘ and Pₙ indicate Pₘᵢₙ and nth aortic pressures during the diastolic phase, respectively, and Pₘᵢₙ indicates the minimum aortic pressure.

The caiFR module is configured to obtain the instantaneous wave-free ratio caiFR through the third mean pressure of aorta P̅ₐ̅₃̅ and the third mean pressure of a distal end of coronary artery stenosis P̅_̅{̅d̅3̅}̅ within the period of wave-free.

The fractional flow reserve caFFR module is configured to obtain fractional flow reserve caFFR through the fourth mean pressure of aorta P̅ₐ̅₄̅ and the fourth mean pressure of a distal end of coronary artery stenosis P̅_̅{̅d̅4̅}̅ in the whole cardiac cycle.

In a third aspect, the disclosure provides a coronary artery analysis system, comprising: the above device for acquiring coronary artery blood vessel evaluation parameters based on an angiographic image.

In a fourth aspect, the disclosure provides a computer storage medium, and when the computer program is executed by a processor, a method for acquiring coronary artery blood vessel evaluation parameters based on an angiographic image is realized.

The beneficial effects brought by the solutions provided by the embodiments of the disclosure at least comprise:

the disclosure provides a method for acquiring coronary artery blood vessel evaluation parameters based on an angiographic image, by acquiring Pₐ-t pressure waveform in an angiographic state in the time domain, and then subjecting the coronary artery two-dimensional angiographic image in the angiographic state to three-dimensional modeling to obtain v-t velocity waveform in the time domain, thereby realizing the measurement of the coronary artery vessel evaluation parameters in a diastolic phase; further, filtering interference data through the Fourier transform improves the accuracy of the coronary artery vessel evaluation parameter measurement.

### BRIEF DESCRIPTION OF DRAWINGS

The drawings described here are configured to provide a further understanding of the disclosure and constitute a part of the disclosure. The exemplary embodiments and descriptions of the disclosure are configured to explain the disclosure, and do not constitute an improper limitation of the disclosure. In the attached drawings:

FIG. 1 is a flow chart of a method for acquiring coronary artery blood vessel evaluation parameters based on an angiographic image of the disclosure;

FIG. 2 is a flowchart of S200 of the disclosure:

FIG. 3 is a flowchart of S300 of the disclosure:

FIG. 4 is a flowchart of S500 of the disclosure:

FIG. 5 is a pressure waveform diagram of a first diastolic phase pressure ratio cadPR of the disclosure;

FIG. 6 is a pressure waveform diagram of a ratio of diastolic phase less than the average pressure caDFR of the disclosure;

FIG. 7 is a pressure waveform diagram of an instantaneous wave-free ratio caiFR of the disclosure;

FIG. 8 is a structural block diagram of an embodiment of the device for acquiring coronary artery blood vessel evaluation parameters based on the angiographic image of the disclosure:

FIG. 9 is a structural block diagram of another embodiment of the device for acquiring coronary artery blood vessel evaluation parameters based on the angiographic image of the disclosure;

FIG. 10 is a schematic structural diagram of the blood pressure acquisition device of the disclosure;

The reference signs are described below:

blood pressure acquisition device 100, main body 110, first power drive device 120, blood pressure acquisition unit 130, first control device 140, fixed block 150, second control device 160, infusion device 170, infusion tube 171, three-dimensional modeling unit 200, blood flow velocity unit 300, pressure drop unit 400, coronary artery blood vessel evaluation parameter unit 500. Fourier transform module 410, pressure drop calculation module 420, Fourier transform inverse module 430, caiFR module 510, cadPR module 520, caDFR module 530, and caFFR module 540.

### DETAILED DESCRIPTION OF PREFERRED EMBODIMENTS

In order to make the objectives, technical solutions and advantages of the present invention clearer, the technical solutions of the present invention will be described dearly and completely in conjunction with specific embodiments of the present invention and the corresponding drawings. Obviously, the described embodiments are only a part of the embodiments of the present invention, rather than all the embodiments. Based on the embodiments of the present invention, all other embodiments obtained by those of ordinary skill in the art without creative work shall fall within the protection scope of the present invention.

Hereinafter, a number of embodiments of the present invention will be disclosed in drawings. For clear description, many practical details will be described in the following description. However, it should be understood that these practical details should not be configured to limit the present invention. In other words, in some embodiments of the present invention, these practical details are unnecessary. In addition, in order to simplify the drawings, some conventionally used structures and components will be shown in simple schematic ways in the drawings.

The existing methods for measuring the evaluation parameters of coronary artery blood vessel are mainly: (1) measurement by pressure guide wire, which is expensive, difficult and risky; (2) measurement through two-dimensional coronary artery angiographic images by obtaining a real-time aortic pressure Pₐ and the measured full-cycle blood flow velocity v, resulting in obtaining only full-cycle coronary artery blood vessel evaluation parameters instead of blood vessel evaluation parameters in a diastolic phase in an angiographic state; and there is no screening of the aortic pressure Pₐ and the blood flow velocity v, resulting in inaccurate blood vessel evaluation parameters.

In order to solve the above problems, a method, device, system and storage medium for acquiring the blood flow of the cardiac superficial aorta.

### Embodiment 1

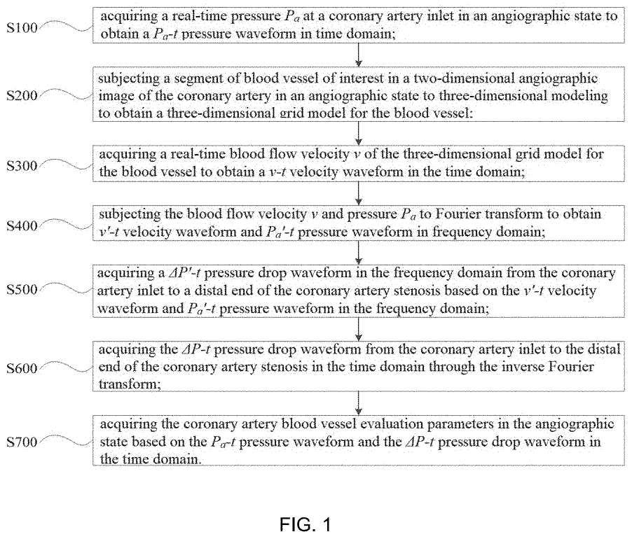

As shown in FIG. 1, the present disclosure provides a method for acquiring coronary artery blood vessel evaluation parameters based on an angiographic image, comprising:

S100, acquiring a real-time pressure Pₐ at a coronary artery inlet in an angiographic state to obtain a Pₐ-t pressure waveform in time domain;

S200, subjecting a segment of blood vessel of interest in a two-dimensional angiographic image of the coronary artery in an angiographic state to three-dimensional modeling to obtain a three-dimensional grid model for the blood vessel;

S300, acquiring a real-time blood flow velocity v of the three-dimensional grid model for the blood vessel to obtain a v-t velocity waveform in the time domain:

S400, subjecting the blood flow velocity v and pressure Pₐ to Fourier transform to obtain v′-t velocity waveform and Pₐ′-t pressure waveform in frequency domain;

S500, acquiring a ΔP′-t pressure drop waveform in the frequency domain from the coronary artery inlet to a distal end of the coronary artery stenosis based on the v′-t velocity waveform and Pₐ′-t pressure waveform in the frequency domain;

S600, acquiring the ΔP-t pressure drop waveform from the coronary artery inlet to the distal end of the coronary artery stenosis in the time domain through the inverse Fourier transform;

S700, acquiring the coronary artery blood vessel evaluation parameters in the angiographic state based on the Pₐ-t pressure waveform and the ΔP-t pressure drop waveform in the time domain.

the disclosure provides a method for acquiring coronary artery blood vessel evaluation parameters based on an angiographic image, by acquiring Pₐ-t pressure waveform in an angiographic state in the time domain, and then subjecting the coronary artery two-dimensional angiographic image in the angiographic state to three-dimensional modeling to obtain v-t velocity waveform in the time domain, thereby realizing the measurement of the coronary artery vessel evaluation parameters in a diastolic phase; further, filtering interference data through the Fourier transform improves the accuracy of the coronary artery vessel evaluation parameter measurement.

### Embodiment 2

S100: acquiring a real-time pressure Pₐ at a coronary artery inlet in an angiographic state to obtain a Pₐ-t pressure waveform in time domain, comprising:

real-time measuring of the pressure Pₐ at the coronary artery inlet through a blood pressure acquisition device connected with a catheter for angiography, and drawing a curve with time to obtain the Pₐ-t pressure waveform in the time domain;

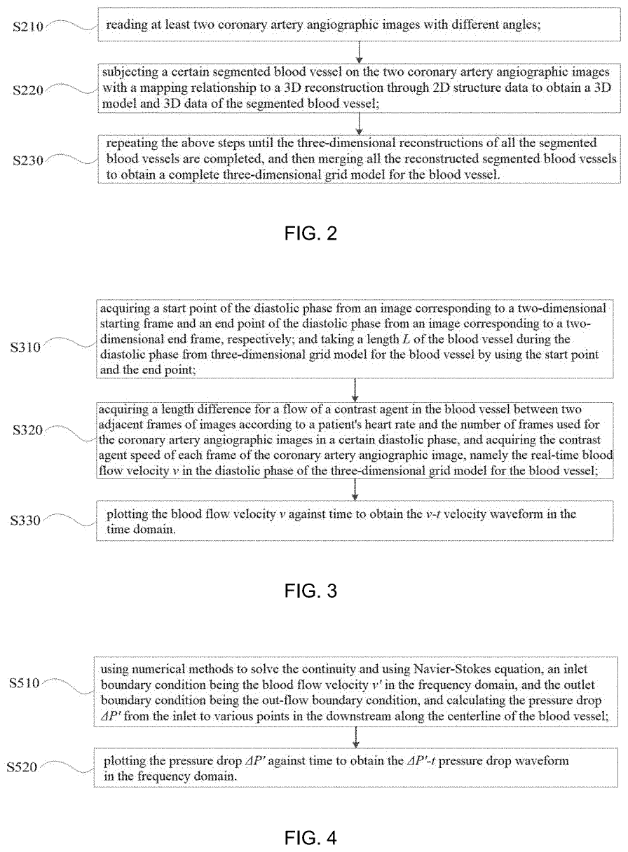

S200: subjecting a segment of blood vessel of interest in a two-dimensional angiographic image of the coronary artery in an angiographic state to three-dimensional modeling to obtain a three-dimensional grid model for the blood vessel, as shown in FIG. 2, comprising:

S210, reading at least two coronary artery angiographic images with different angles; further comprising: denoising the coronary artery angiographic images; the position shooting angle of the two coronary artery angiographic images being greater than or equal to 30°;

comprising: static noise and dynamic noise; static noise is noise that is static in time, such as ribs in the chest cavity; dynamic noise is noise that changes in time, such as part of lung tissue and part of heart tissue; and comprising: through gray-level histogram analysis, using threshold to further denoise.

S220, subjecting a certain segmented blood vessel on the two coronary artery angiographic images with a mapping relationship to a 3D reconstruction through 2D structure data to obtain a 3D model and 3D data of the segmented blood vessel;

S230: repeating the above steps until the three-dimensional reconstructions of all the segmented blood vessels are completed, and then merging all the reconstructed segmented blood vessels to obtain a complete three-dimensional grid model for the blood vessel;

S300: acquiring a real-time blood flow velocity v of the three-dimensional grid model for the blood vessel to obtain a v-t velocity waveform in the time domain, as shown in FIG. 3, comprising:

S310: acquiring a start point of the diastolic phase from an image corresponding to a two-dimensional starting frame and an end point of the diastolic phase from an image corresponding to a two-dimensional end frame, respectively; and taking a length L of the blood vessel during the diastolic phase from three-dimensional grid model for the blood vessel by using the start point and the end point, comprising: removing interfering blood vessels, extracting the coronary centerline and diameter of the blood vessel of interest along the extension direction of the coronary artery;

S320, acquiring a length difference for a flow of a contrast agent in the blood vessel between two adjacent frames of images according to a patient's heart rate and the number of frames used for the coronary artery angiographic images in a certain diastolic phase, and acquiring the contrast agent speed of each frame of the coronary artery angiographic image, namely the real-time blood flow velocity v of the three-dimensional grid model for the blood vessel, the specific formula being as follows:

(1) If the real-time blood flow velocity v is the blood flow velocity in the diastolic phase, then

K=t/T;

$$v = Δ L / [ \frac{K ( 60 / H )}{y} ] ;$$

wherein, ΔL represents the length difference for the flow of the contrast agent in the blood vessel between two adjacent frames of images, H represents the patient's heart rate with a unit of bets/min; y represents the number of frames of the coronary artery angiographic images in the diastolic phase, wherein t represents the time for the diastolic phase in the Pₐ-t pressure waveform, T represents the time for the whole cardiac cycle in the Pₐ-t pressure waveform:

(2) If the real-time blood flow velocity v is the blood flow velocity of the whole cardiac cycle, then

$$v = Δ L / [ \frac{( 60 / H )}{x} ] ;$$

wherein, ΔL represents the length difference for the flow of the contrast agent in the blood vessel between two adjacent frames of images, H represents the patient's heart rate with a unit of beats/min, and x represents the number of the frames of the coronary artery angiographic images within the cardiac cycle;

S330: plotting the blood flow velocity v against time to obtain the v-t velocity waveform in the time domain;

S400: subjecting Fourier transform to the blood flow velocity v and pressure Pₐ to obtain v′-t velocity waveform and Pₐ′-t pressure waveform in frequency domain;

S500, acquiring a ΔP′-t pressure drop waveform in the frequency domain from the coronary artery inlet to the distal end of the coronary artery stenosis based on the v′-t velocity waveform and Pₐ′-t pressure waveform in the frequency domain, as shown in FIG. 4, comprising:

S510, using numerical methods to solve the continuity and using Navier-Stokes equation to calculate the pressure drop ΔP′ from the inlet to various points in the downstream along the center line of blood vessels, an inlet boundary condition being the blood flow velocity v′ in the frequency domain, and an outlet boundary condition being the out-flow boundary condition;

wherein, the continuity and Navier-Stokes equation are:

$$▽ \cdot \vec{V} = 0 ; \\ ρ \frac{\partial \vec{V}}{\partial t} + ρ \vec{V} \cdot ▽ \vec{V} = - ▽ P + ▽ \cdot {μ ( ▽ \vec{V} + ▽ \vec{V} )}^{T} ) ;$$

V̅, P, ρ, μ represent a coronary artery blood flow velocity, a pressure, a blood flow density, and a blood flow viscosity, respectively;

S520, plotting the pressure drop ΔP′ against time to obtain the ΔP-t pressure drop waveform in the frequency domain;

S600: acquiring the ΔP-t pressure drop waveform from the coronary artery inlet to the distal end of the coronary artery stenosis in the time domain through the inverse Fourier transform;

S700, acquiring the coronary artery blood vessel evaluation parameters in the angiographic state based on the Pₐ-t pressure waveform and the ΔP-t pressure drop waveform in the time domain, comprising: instantaneous wave-free ratio caiFR in the angiographic state, a first diastolic phase pressure ratio in the angiographic state cadPR, a ratio of diastolic phase less than the average pressure in the angiographic state caDFR, and fractional flow reserve caFFR, then:

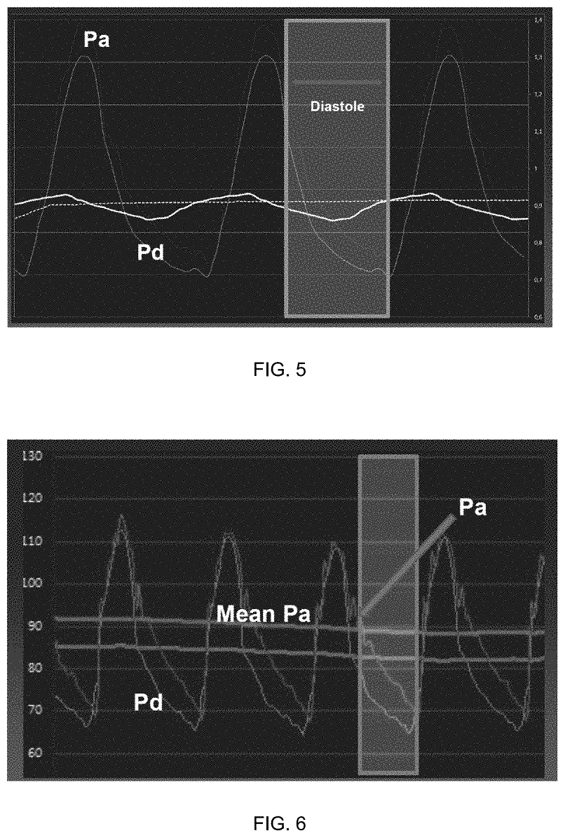

(1) as shown in FIG. 5, the first diastolic phase pressure ratio cadPR is obtained through the first mean pressure of aorta P̅ₐ̅₁̅ in the full diastolic phase (namely Diastole strip area in FIG. 5) and the first mean pressure of a distal end of artery stenosis P̅_̅{̅d̅1̅}̅, namely: cadPR=P̅_̅{̅d̅1̅}̅/P̅ₐ̅₁̅;

(2) as shown in FIG. 6, the ratio of diastolic phase less than the average pressure caDFR is obtained through the second mean pressure of aorta P̅ₐ̅₂̅ and the second mean pressure of a distal end of coronary artery stenosis P̅_̅{̅d̅2̅}̅ within an interval from Pₐ<P̅ₐ̅₁̅, to the aortic pressure Pₙ=Pₘᵢₙ, wherein Pₘ and Pₙ indicate mth and nth aortic pressures during the diastolic phase, respectively, namely: caDFR=P̅_̅{̅d̅2̅}̅/P̅ₐ̅₂̅, Pₘᵢₙ indicates the minimum aortic pressure;

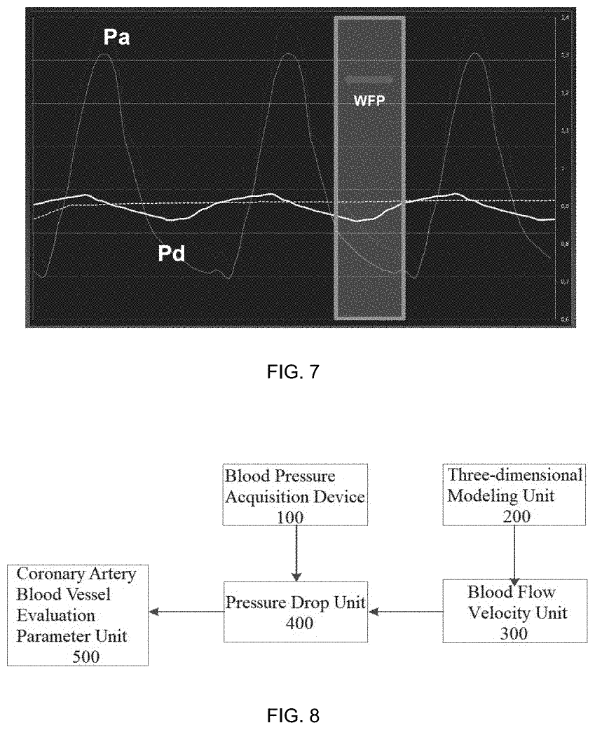

(3) as shown in FIG. 7, the instantaneous wave-free ratio caiFR is obtained through the third mean pressure of aorta P̅ₐ̅₃̅ and the third mean pressure of a distal end of coronary artery stenosis P̅_̅{̅d̅3̅}̅ within the period of wave-free, namely: caiFR=P̅_̅{̅d̅3̅}̅/P̅ₐ̅₃̅;

The wave-free is defined as a certain period of time in the diastolic phase, which is called the wave-free period.

The calculation time for the instantaneous wave-free period is calculated from 25% of the time after the start of the wave-free period to 5 ms before the start of a systolic phase.

(4) The fractional flow reserve caFFR is obtained through the fourth mean pressure of aorta P̅ₐ̅₄̅ and the fourth mean pressure of a distal end of coronary artery stenosis P̅_̅{̅d̅4̅}̅ in the whole cardiac cycle, namely:

caFFR=P̅_̅{̅d̅4̅}̅/P̅ₐ̅₄̅.

In another embodiment of the present disclosure, the blood flow velocity v in S300 is calculated by v=ΔL/fps, where ΔL represents the length difference between the blood vessels in which the contrast agents of two adjacent frames of image are flowing, and fps represents the number of frames transmitted per second.

### Embodiment 3

As shown in FIG. 8, the present disclosure provides an device for acquiring coronary artery blood vessel evaluation parameters based on an angiographic image and the device is used in the method for acquiring coronary artery blood vessel evaluation parameters based on the angiographic image, and the device comprises: a blood pressure acquisition device 100, and a three-dimensional modeling unit 200, a blood flow velocity unit 300, a pressure drop unit 400, and a coronary artery blood vessel evaluation parameter unit 500 connected in sequence, and the blood pressure acquisition device 100 is connected to the pressure drop unit 400; the blood pressure acquisition device 100 is connected to an external catheter for angiography and for acquiring a real-time pressure Pₐ at a coronary artery inlet in an angiographic state to obtain a Pₐ-t pressure waveform in time domain; the three-dimensional modeling unit 200 is configured for reading the coronary artery angiographic image in the angiographic state, and subjecting a segment of blood vessel of interest in the image to three-dimensional modeling to obtain a three-dimensional grid model for the blood vessel; the blood flow velocity unit 300 is configured for receiving the three-dimensional grid model for the blood vessel sent by the three-dimensional modeling unit 200 and acquiring a real-time blood flow velocity v of the segment of blood vessel of interest to obtain v-t velocity waveform in the time domain; as shown in FIG. 6, the pressure drop unit 400 also comprises: a Fourier transform module 410, a pressure drop calculation module 420, and a Fourier transform inverse module 430 sequentially connected, and a Fourier transform module 410 is configured to receive the v-t velocity waveform and the Pₐ-t pressure waveform in the time domain sent by the blood flow velocity unit 300 and the blood pressure acquisition device 100, respectively, and the blood flow velocity v and pressure Pₐ is subjected to Fourier transform to obtain v′-t velocity waveform and Pₐ′-t pressure waveform in frequency domain; the pressure drop calculation module 420 is then configured to acquire a ΔP′-t pressure drop waveform in the frequency domain from the coronary artery inlet to the distal end of the coronary artery stenosis based on the v′-t velocity waveform and Pₐ′-t pressure waveform in the frequency domain sent by the Fourier transform module 410; the Fourier transform inverse module 430 is configured to acquire the ΔP-t pressure drop waveform from the coronary artery inlet to the distal end of the coronary artery stenosis in the time domain through the inverse Fourier transform according to the ΔP′-t pressure drop waveform sent by the pressure drop calculation module 420; the coronary artery blood vessel evaluation parameter unit 500 is configured for receiving the Pₐ-t pressure waveform and the ΔP-t pressure drop waveform in the time domain sent by the blood pressure acquisition device 100 and the pressure drop unit 400 to acquire the coronary artery blood vessel evaluation parameters.

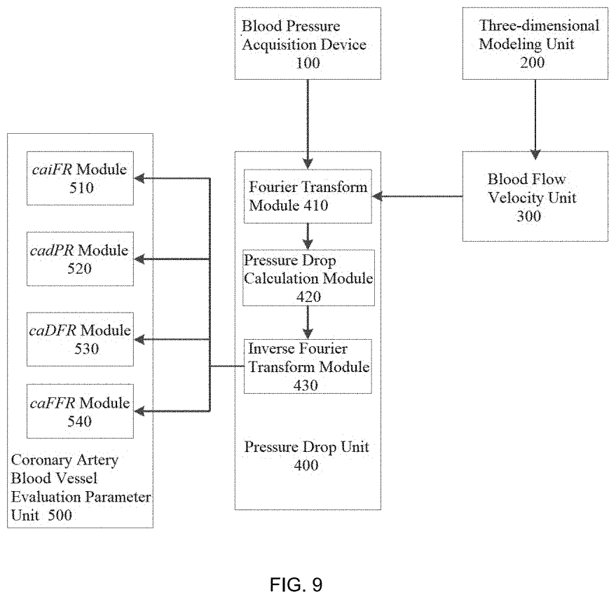

In an embodiment of the present disclosure, as shown in FIG. 9, the coronary artery blood vessel evaluation parameter unit 500 further comprises: a caiFR module 510, a cadPR module 520, a caDFR module 530, and a caFFR module 540, which are all connected to the Fourier transform inverse module 430. The cadPR module 520 is configured to select the waveform in the full diastolic phase from the Pₐ-t pressure waveform and the ΔP-t pressure drop waveform in the time domain sent by the pressure drop unit 400, and obtain the first diastolic phase pressure ratio cadPR through the first mean pressure of aorta P̅ₐ̅₁̅ in the full diastolic phase and the first mean pressure of a distal end of artery stenosis P̅_̅{̅d̅1̅}̅; the caDFR module 530 is configured to select an interval waveform from Pₐ<P̅ₐ̅₁̅ to the aortic pressure Pₙ=Pₘᵢₙ from the Pₐ-t pressure waveform and the ΔP-t pressure drop waveform in the time domain sent by the pressure drop unit 400, and obtain the ratio of a diastolic phase less than the average pressure caDFR through the second mean pressure of aorta P̅ₐ̅₂̅ and the second mean pressure of a distal end of coronary artery stenosis P̅_̅{̅d̅2̅}̅, wherein Pₘ and Pₙ indicate mth and nth aortic pressures during the diastolic phase, respectively: the caiFR module 510 is configured to select the waveform in the wave-free period from the Pₐ-t pressure waveform and the ΔP-t pressure drop waveform in the time domain sent by the pressure drop unit 400, and obtain the instantaneous wave-free ratio caiFR through the third mean pressure of aorta P̅ₐ̅₃̅ and the third mean pressure of a distal end of coronary artery stenosis P̅_̅{̅d̅3̅}̅ within the period of wave-free; the fractional flow reserve caFFR is configured to select all data from the Pₐ-t pressure waveform and the ΔP-t pressure drop waveform in the time domain sent by the pressure drop unit 400 as the whole cardiac cycle data, and obtain fractional flow reserve caFFR through the fourth mean pressure of aorta P̅ₐ̅₄̅ and the fourth mean pressure of a distal end of coronary artery stenosis P̅_̅{̅d̅4̅}̅ in the whole cardiac cycle.

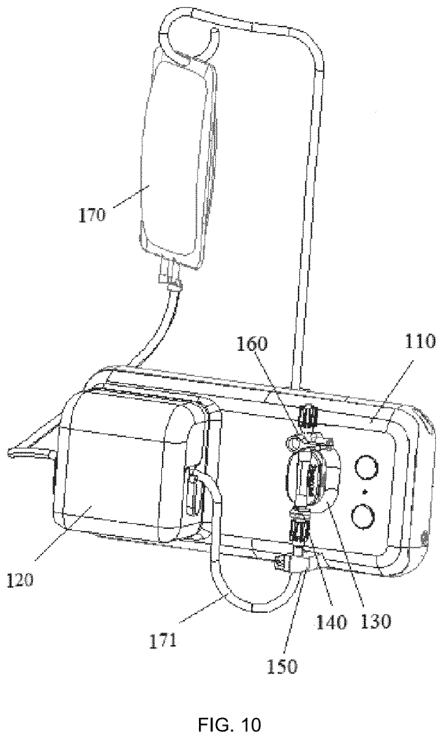

In an embodiment of the present disclosure, as shown in FIG. 10, the blood pressure acquisition device 100 comprises: a main body 110, and a first power drive device 120, a blood pressure acquisition unit 130, and a first control device 140 that are all connected to the main body 110; the main body 110 is configured to control whether the first power drive device 120, the blood pressure acquisition unit 130, and the first control device 140 start working; the blood pressure acquisition unit 130 is connected to the second control device 160, and the second control device 160 is connected to an external interventional device; the second control device 160 is configured for zeroing the blood pressure acquisition unit 130 and for controlling whether the blood pressure acquisition unit 130 is in communication with the external interventional device; the first power drive device 120 is arranged on the main body 110, and the first power drive device 120 is connected to the external infusion device 170, and the first power drive device 120 is configured to drive the liquid flow of the external infusion device 170; the blood pressure acquisition unit 130 is arranged on the main body 110, and the blood pressure acquisition unit 130 is connected to the first control device 140 and the external interventional device, and the blood pressure acquisition unit 130 is configured to collect the invasive arterial pressure; the first control device 140 is fixed on the main body 110 through the fixing block 150, etc.; the first control device 140 is connected to the external infusion device 170 for controlling the liquid flow direction of the infusion device 170, which causes the liquid to flow from the infusion device 170 to the first control device 140. The present disclosure provides a blood pressure acquisition device, and by setting the first power drive device 120, the blood pressure acquisition unit 130, and the first control device 140 on the main body 110 and by opening the first control device 140, the blood pressure acquisition unit 130 is in communication with the external infusion device 170 and atmosphere at the same time, so that the first power drive device 120 drives the liquid flow inside the infusion tube 7100 on the external infusion device 170, thereby realizing automatic discharge without manual discharge, which is simple and convenient to operate; the automatic zero calibration of the blood pressure acquisition unit 130 can be realized by setting the second control device 160; because the height change of the operating bed will affect the measurement of the invasive arterial pressure, the height of the blood pressure acquisition device needs to be changed during an operation, and there is no need to repeat the zero calibration many times, and the operation is simple and the measurement is accurate.

In a third aspect, the present disclosure provides a coronary artery analysis system, comprising: the above device for acquiring coronary artery blood vessel evaluation parameters based on an angiographic image.

In a fourth aspect, the present disclosure provides a computer storage medium, and when the computer program is executed by a processor, the above method for acquiring coronary artery blood vessel evaluation parameters based on an angiographic image is realized.

Those skilled in the art know that various aspects of the present invention can be implemented as a system, a method, or a computer program product. Therefore, each aspect of the present invention can be specifically implemented in the following forms, namely: complete hardware implementation, complete software implementation (comprising firmware, resident software, microcode, etc.), or a combination of hardware and software implementations, Here can be collectively referred to as “circuit”, “module” or “system”. In addition, in some embodiments, various aspects of the present invention may also be implemented in the form of a computer program product in one or more computer-readable media, and the computer-readable medium contains computer-readable program code. Implementation of methods and/or systems of embodiments of the present invention may involve performing or completing selected tasks manually, automatically, or a combination thereof.

For example, hardware for performing selected tasks according to an embodiment of the present invention may be implemented as a chip or a circuit. As software, selected tasks according to an embodiment of the present invention can be implemented as a plurality of software instructions executed by a computer using any suitable operating system. In an exemplary embodiment of the present invention, a data processor performs one or more tasks according to an exemplary embodiment of a method and/or system as herein, such as a computing platform for executing a plurality of instructions. Optionally, the data processor includes a volatile memory for storing instructions and/or data and/or a non-volatile memory for storing instructions and/or data, for example, a magnetic hard disk and/or a removable medium. Optionally, a network connection is also provided. Optionally, a display and/or user input device, such as a keyboard or mouse, is also provided.

Any combination of one or more computer readable can be utilized. The computer-readable medium may be a computer-readable signal medium or a computer-readable storage medium. The computer-readable storage medium may be, for example, but not limited to, an electrical, magnetic, optical, electromagnetic, infrared, or semiconductor system, apparatus, or device, or any combination of the above. More specific examples (non-exhaustive list) of computer-readable storage media would include the following:

Electrical connection with one or more wires, portable computer disk, hard disk, random access memory (RAM), read only memory (ROM), erasable programmable read only memory (EPROM or flash memory), optical fiber, portable compact disk read only memory (CD-ROM), optical storage device, magnetic storage device, or any suitable combination of the above. In this document, the computer-readable storage medium can be any tangible medium that contains or stores a program, and the program can be used by or in combination with an instruction execution system, apparatus, or device.

The computer-readable signal medium may include a data signal propagated in baseband or as a part of a carrier wave, and computer-readable program code is carried therein. This propagated data signal can take many forms, comprising but not limited to electromagnetic signals, optical signals, or any suitable combination of the above. The computer-readable signal medium may also be any computer-readable medium other than the computer-readable storage medium. The computer-readable medium may send, propagate, or transmit the program use by or in combination with the instruction execution system, apparatus, or device.

The program code contained on the computer-readable medium can be transmitted by any suitable medium, comprising (but not limited to) wireless, wired, optical cable. RF, etc., or any suitable combination of the foregoing.

For example, any combination of one or more programming languages can be configured to write computer program codes for performing operations for various aspects of the present invention, comprising object-oriented programming languages such as Java, Smalltalk, C++, and conventional process programming languages, such as “C” programming language or similar programming language. The program code may be executed entirely on the user's computer, partly on the users computer, executed as an independent software package, partly on the users computer and partly executed on a remote computer, or entirely executed on the remote computer or server. In the case of a remote computer, the remote computer can be connected to the users computer through any kind of network comprising a local area network (LAN) or a wide area network (WAN), or it can be connected to an external computer (for example, using an Internet service provider to pass Internet connection).

It should be understood that each block of the flowchart and/or block diagram and the combination of each block in the flowchart and/or block diagram may be implemented by computer program instructions. These computer program instructions can be provided to the processors of general-purpose computers, special-purpose computers, or other programmable data processing devices, thereby producing a machine that makes these computer program instructions when executed by the processors of the computer or other programmable data processing devices, a device that implements the functions/actions specified in one or more blocks in the flowchart and/or block diagram is produced.

It is also possible to store these computer program instructions in a computer-readable medium. These instructions make computers, other programmable data processing devices, or other devices work in a specific manner, so that the instructions stored in the computer-readable medium generate an article of manufacture that implements instructions for functions/actions specified in one or more blocks in the flowchart and/or block diagram.

Computer program instructions can also be loaded onto a computer (for example, a coronary artery analysis system) or other programmable data processing equipment to cause a series of operation steps to be performed on the computer, other programmable data processing equipment or other equipment to produce a computer-implemented process, causing instructions executed on a computer, other programmable device or other equipment to provide a process for implementing the functions/actions specified in the flowchart and/or one or more block diagrams.

The above specific examples of the present invention further describe the objectives, technical solutions and beneficial effects of the present invention in detail. It should be understood that the above are only specific embodiments of the present invention and are not intended to limit the present invention. Within the spirit and principle of the present invention, any modifications, equivalent substitutions, improvements, etc., shall be included in the protection scope of the present invention.

What is claimed is:

1. A method for acquiring coronary artery blood vessel evaluation parameters based on an angiographic image, characterized in that, comprising the following steps:
acquiring a real-time pressure Pₐ at a coronary artery inlet in an angiographic state to obtain a Pₐ-t pressure waveform in time domain;
subjecting a segment of blood vessel of interest in a two-dimensional angiographic image of the coronary artery in an angiographic state to three-dimensional modeling to obtain a three-dimensional grid model for the blood vessel;
acquiring a real-time blood flow velocity v of the three-dimensional grid model for the blood vessel to obtain a v-t velocity waveform in the time domain;
subjecting the blood flow velocity v and pressure Pₐ to Fourier transform to obtain v′-t velocity waveform and Pₐ′-t pressure waveform in frequency domain;
acquiring a ΔP′-t pressure drop waveform in the frequency domain from the coronary artery inlet to a distal end of the coronary artery stenosis based on the v′-t velocity waveform and Pₐ′-t pressure waveform in the frequency domain;
acquiring the ΔP-t pressure drop waveform from the coronary artery inlet to the distal end of the coronary artery stenosis in the time domain through the inverse Fourier transform;
acquiring the coronary artery blood vessel evaluation parameters in the angiographic state based on the Pₐ-t pressure waveform and the ΔP-t pressure drop waveform in the time domain.

2. The method for acquiring the coronary artery blood vessel evaluation parameters based on the angiographic image according to claim 1, wherein the blood vessel evaluation parameters comprise: instantaneous wave-free ratio caiFR in the angiographic state, a first diastolic phase pressure ratio in the angiographic state cadPR and a ratio of diastolic phase less than the average pressure in the angiographic state caDFR; wherein,
the first diastolic phase pressure ratio cadPR is obtained through the first mean pressure of aorta P̅ₐ̅₁̅ in the full diastolic phase and the first mean pressure of a distal end of artery stenosis P̅_̅{̅d̅1̅}̅;
the ratio of diastolic phase less than the average pressure caDFR is obtained through the second mean pressure of aorta P̅ₐ̅₂̅, and the second mean pressure of a distal end of coronary artery stenosis P̅_̅{̅d̅2̅}̅, within an interval from Pₘ<P̅ₐ̅₁̅ to the aortic pressure Pₙ=Pₘᵢₙ wherein Pₘ and Pₙ indicate m^{th} and n^{th} aortic pressures during the diastolic phase, respectively, and Pₘᵢₙ indicates the minimum aortic pressure;
the instantaneous wave-free ratio caiFR is obtained through the third mean pressure of aorta P̅ₐ̅₃̅ and the third mean pressure of a distal end of coronary artery stenosis P̅_̅{̅d̅3̅}̅, within the period of wave-free.

3. The method for acquiring the coronary artery blood vessel evaluation parameters based on the angiographic image according to claim 2, wherein calculation formulas of the instantaneous wave-free ratio caiFR, the first diastolic pressure ratio cadPR and the ratio of diastolic phase less than the average pressure caDFR each are:
cadPR=P̅_̅{̅d̅1̅}̅/P̅ₐ̅₁̅;caDFR=P̅_̅{̅d̅2̅}̅/P̅ₐ̅₂̅;caiFR=P̅_̅{̅d̅3̅}̅/P̅ₐ̅₃̅.

4. The method for acquiring the coronary artery blood vessel evaluation parameters based on an angiographic image according to claim 1, wherein the blood vessel evaluation parameters further comprise: fractional flow reserve caFFR; wherein,
fractional flow reserve caFFR is obtained through the fourth mean pressure of aorta P̅ₐ̅₄̅ and the fourth mean pressure of a distal end of coronary artery stenosis P̅_̅{̅d̅4̅}̅ in the whole cardiac cycle, namely:
caFFR=P̅_̅{̅d̅4̅}̅/P̅ₐ̅₄̅.

5. The method for acquiring the coronary artery blood vessel evaluation parameters based on the angiographic image according to claim 1, wherein the step for acquiring the real-time pressure Pₐ at the coronary artery inlet in the angiographic state to obtain the Pₐ-t pressure waveform in the time domain comprises: real-time measuring the pressure Pₐ at the coronary artery inlet through a blood pressure acquisition device connected with a catheter for angiography, and drawing a curve with time to obtain the Pₐ-t pressure waveform in the time domain.

6. The method for acquiring the coronary artery blood vessel evaluation parameters based on the angiographic image according to claim 5, wherein the step for subjecting the segment of the blood vessel of interest in the two-dimensional angiographic image of the coronary artery in the angiographic state to three-dimensional modeling to obtain the three-dimensional grid model for the blood vessel comprises:
reading at least two coronary artery angiographic images with different angles;
subjecting a certain segmented blood vessel on the two coronary angiographic images with a mapping relationship to a 3D reconstruction through 2D structure data to obtain a 3D model and 3D data of the segmented blood vessel;
repeating the above steps until the three-dimensional reconstructions of all the segmented vessels are completed, and then merging all the reconstructed segmented vessels to obtain a complete three-dimensional vessel grid model.

7. The method for acquiring coronary artery blood vessel evaluation parameters based on the angiographic image according to claim 6, wherein the step for acquiring the real-time blood flow velocity v of the three-dimensional grid model for the blood vessel to obtain the v-t velocity waveform in the time domain comprises:
acquiring a start point of the diastolic phase from an image corresponding to a two-dimensional starting frame and an end point of the diastolic phase from an image corresponding to a two-dimensional end frame, respectively; and taking a length L of the blood vessel in the diastolic phase from three-dimensional grid model for the blood vessel by using the start point and the end point;
acquiring the length difference between the blood vessels in which the contrast agents of two adjacent frames of images are flowing according to a patient's heart rate and the number of frames the coronary artery angiographic images passing through in a certain diastolic phase, and acquiring the contrast agent speed of each frame of the coronary artery angiographic image, namely the real-time blood flow velocity v in the diastolic phase of the three-dimensional grid model for the blood vessel, the specific formula being as follows:
$$v = Δ L / [ \frac{K ( 60 / H )}{y} ] ;$$
$$K = t / T ,$$
wherein, ΔL represents the length difference for the flow of the contrast agent in the blood vessel between two adjacent frames of images, H represents the patients heart rate with a unit of beats/min; y represents the number of frames of the coronary artery angiographic images in the diastolic phase, wherein t represents the time for the diastolic phase in the Pₐ-t pressure waveform, T represents the time for the whole cardiac cycle in the Pₐ-t pressure waveform;
plotting the blood flow velocity v against time to obtain the v-t velocity waveform in the time domain in the diastolic phase.

8. The method for acquiring coronary artery blood vessel evaluation parameters based on an angiographic image according to claim 1, wherein the step for acquiring the real-time blood flow velocity v of the three-dimensional grid model for blood vessel to obtain the v-t velocity waveform in the time domain comprises:
acquiring a start point of the cardiac cycle from an image corresponding to a two-dimensional starting frame and an end point of the cardiac cycle from an image corresponding to a two-dimensional end frame, respectively; and taking a length L of the blood vessel within one cardiac cycle from three-dimensional grid model for the blood vessel by using the start point and the end point:
acquiring a length difference for a flow of a contrast agent in the blood vessel between two adjacent frames of images according to a patients heart rate and the number of frames used for the coronary artery angiographic images in a certain cardiac cycle, and acquiring the contrast agent speed of each frame of the coronary artery angiographic image, namely the real-time blood flow velocity v in the whole cardiac cycle of the three-dimensional grid model for the blood vessel, the specific formula being as follows:
$$v = Δ L / [ \frac{( 60 / H )}{x} ] ;$$
wherein, ΔL represents the length difference for the flow of the contrast agent in the blood vessel between two adjacent frames of images, H represents the patient's heart rate with a unit of beats/min, and x represents the number of the frames of the coronary artery angiographic images within the cardiac cycle;
plotting the blood flow velocity v against time to obtain the v-t velocity waveform in the time domain in the cardiac cycle.

9. The method for acquiring coronary artery blood vessel evaluation parameters based on an angiographic image according to claim 1, wherein the step for acquiring a real-time blood flow velocity v of the three-dimensional grid model for blood vessel comprises:
v=ΔL/fps;
wherein, ΔL represents the length difference for the flow of the contrast agent in the blood vessel between two adjacent frames of images, and fps represents the number of frames transmitted per second.

10. The method for acquiring coronary artery blood vessel evaluation parameters based on an angiographic image according to claim 1, wherein the step for acquiring the ΔP′-t pressure drop waveform in the frequency domain from the coronary artery inlet to the distal end of the coronary artery stenosis based on the v′-t velocity waveform and Pₐ′-t pressure waveform in the frequency domain comprises:
using numerical methods to solve the continuity and using Navier-Stokes equation to solve the pressure drop ΔP′ from the coronary artery inlet to the distal end of the coronary artery stenosis, the specific formula being as follows:
$$▽ \cdot \vec{V} = 0 ; \\ ρ \frac{\partial \vec{V}}{\partial t} + ρ \vec{V} \cdot ▽ \vec{V} = - ▽ P + ▽ \cdot {μ ( ▽ \vec{V} + ▽ \vec{V} )}^{T} ) ;$$
wherein, V̅, P, ρ, μ represent a coronary artery blood flow velocity, a pressure, a blood flow density, and a blood flow viscosity, respectively;
an inlet boundary condition being the blood flow velocity v′ in the frequency domain, and the outlet boundary condition being the out-flow boundary condition;
calculating the pressure drop ΔP′ from the inlet to various points in the downstream along the centerline of the blood vessel;
plotting the pressure drop ΔP′ against time to obtain the ΔP-t pressure drop waveform in the frequency domain.

11. A device for acquiring coronary artery blood vessel evaluation parameters based on an angiographic image, which is used in the method for acquiring coronary artery blood vessel evaluation parameters based on an angiographic image according to claim 10, characterized in that, comprising: a blood pressure acquisition device; and a three-dimensional modeling unit, a blood flow velocity unit, a pressure drop unit, and a coronary artery blood vessel evaluation parameter unit connected in sequence, and the blood pressure acquisition device being connected to the pressure drop unit;
the blood pressure acquisition device is connected with an external catheter for angiography and configured to acquire a real-time pressure Pₐ at a coronary artery inlet in an angiographic state to obtain a Pₐ-t pressure waveform in time domain;
the three-dimensional modeling unit is configured to read the coronary artery angiographic image in the angiographic state and subjected a segment of blood vessel of interest in the image to three-dimensional modeling to obtain a three-dimensional grid model for the blood vessel:
the blood flow velocity unit is configured to receive the three-dimensional grid model for the blood vessel sent by the three-dimensional modeling unit and acquired a real-time blood flow velocity v of the segment of blood vessel of interest to obtain v-t velocity waveform in the time domain;
the pressure drop unit is configured to receive the v-t velocity waveform and the Pₐ′-t pressure waveform in the time domain sent by the blood flow velocity unit and the blood pressure acquisition device, respectively, and subject the blood flow velocity v and pressure Pₐ to Fourier transform to obtain v′-t velocity waveform and Pₐ′-t pressure waveform in frequency domain; acquiring a ΔP′-t pressure drop waveform in the frequency domain from the coronary artery inlet to a distal end of the coronary artery stenosis based on the v′-t velocity waveform and Pₐ′-t pressure waveform in the frequency domain; acquiring the ΔP-t pressure drop waveform from the coronary artery inlet to the distal end of the coronary artery stenosis in the time domain through the inverse Fourier transform;
the coronary artery blood vessel evaluation parameter unit is configured to receive the Pₐ-t pressure waveform and the ΔP-t pressure drop waveform in the time domain sent by the blood pressure acquisition device and the pressure drop unit, and acquire the coronary artery blood vessel evaluation parameters.

12. The device for acquiring coronary artery blood vessel evaluation parameters based on an angiographic image according to claim 11, wherein the coronary artery vessel evaluation parameter unit further comprises: a caiFR module, a cadPR module, a caDFR module, and a caFFR module;
the cadPR module is configured to obtain the first diastolic phase pressure ratio cadPR through the first mean pressure of aorta P̅ₐ̅₁̅ in the full diastolic phase and the first mean pressure of a distal end of coronary artery stenosis P̅_̅{̅d̅1̅}̅;
the caDFR module is configured to obtain the ratio of a diastolic phase less than the average pressure caDFR through the second mean pressure of aorta P̅ₐ̅₁̅ and the second mean pressure of a distal end of coronary artery stenosis P̅_̅{̅d̅2̅}̅ within an interval from Pₘ<P̅ₐ̅₁̅ to the aortic pressure Pₙ=Pₘᵢₙ, wherein Pₘ and Pₙ indicate Pₘᵢₙ and nth aortic pressures during the diastolic phase, respectively, and Pₘᵢₙ indicates the minimum aortic pressure;
the caiFR module is configured to obtain the instantaneous wave-free ratio caiFR through the third mean pressure of aorta P̅ₐ̅₃̅ and the third mean pressure of a distal end of coronary artery stenosis P̅_̅{̅d̅3̅}̅ within the period of wave-free;
the fractional flow reserve caFFR module is configured to obtain fractional flow reserve caFFR through the fourth mean pressure of aorta P̅ₐ̅₄̅ and the fourth mean pressure of a distal end of coronary artery stenosis P̅_̅{̅d̅4̅}̅ in the whole cardiac cycle.

13. A coronary artery analysis system, characterized in that, comprising: the device for acquiring coronary artery blood vessel evaluation parameters based on an angiographic image according to claim 11.

14. A computer storage medium, characterized in that, when a computer program is executed by a processor, the method for acquiring coronary artery blood vessel evaluation parameters based on an angiographic image according to claim 1 is realized.
